SEQUENCE LISTING (41 sequences)

SEQ ID NO: 1 - protein, 125 aa, Homo sapiens
  MISC_FEATURE (1)..(125): Vh1 heavy chain variable region
QVQLLVQSGAEVKKPGASVKVSCKASGYTFTXWVRQAPGQGLEWMGXRVTMTRDTSTSTAYMELSSLRSEDTAVYYCARXWGQGTLVTVSSGSTKGPSVFPLAPSSKSTSGGTAALGCLVKDYFP

SEQ ID NO: 2 - protein, 124 aa, Homo sapiens
  MISC_FEATURE (1)..(124): Vh2 heavy chain variable region
QITLKESGPALVKPTQTLTLTCTFSGFSLSXWIRQPPGKALEWLAXRLTITKDTSKNQVVLTMTNMDPVDTATYYCARXWGQGTLVTVSSASPTSPKVFPLSLSSKSTSGGTAALGCLVKDYFP

SEQ ID NO: 3 - protein, 100 aa, Homo sapiens
  MISC_FEATURE (1)..(100): Vh3a heavy chain variable region
EVVQLVESGGGLVQPGGSLRLSCAASGFTFSXWVRQAPGKGLEWVSXRFTISRDNSKNTLYLQMNSLRAEDTAVYYCARXWGQGTLVTVSSGSTKAPSVF

SEQ ID NO: 4 - protein, 102 aa, Homo sapiens
  MISC_FEATURE (1)..(102): Vh3b heavy chain variable region
EVQLVESGGGLVKPGGSLRLSCAASGFTFSXWVRQAPGKGLEWVGXRFTISRDDSKNTLYLQMNSLKTEDTAVYYCTTXWGQGTLVTVSSASTKGPSVFPLA

SEQ ID NO: 5 - protein, 101 aa, Homo sapiens
  MISC_FEATURE (1)..(101): Vh3c heavy chain variable region
EVQLVESGGGLVQPGRSLRLSCTASGFTFGXWVRQAPGKGLEWVGXRFTISRDDSKSIAYLQMNSLKTEDTAVYYCTRNXWGQGTLVTVSSGSTKGPSVLP

SEQ ID NO: 6 - protein, 108 aa, Homo sapiens
  MISC_FEATURE (1)..(108): Vh4 heavy chain variable region
QVQLQESGPGLVKPSETLSLTCTVSGGSSISSSXWIRQPPGKGLEWIGXRVTISVDTSKNQFSLKLSSVTAADTAVYYCARXWGQGTLVTVSSAPTKAPDVFPIISGC

SEQ ID NO: 7 - protein, 132 aa, Homo sapiens
  MISC_FEATURE (1)..(132): Vh5 heavy chain variable region
EEVQLVQSGAEVKKPGESLKISCKGSGYSFTXWVRQMPGKGLEWMGXQVTISADKSISTAYLQWSSLKASDTAMYYCARXWGQGTLVTVSSASTKAPSVFPLVSCENSPSDTSSVAVGCLAQDFLPDSITFS

SEQ ID NO: 8 - protein, 125 aa, Homo sapiens
  MISC_FEATURE (1)..(125): Vh6 heavy chain variable region
QVQLQQSGPGLVKPSQTLSLTCAISGDSVSXWIRQSPSRGLEWLGXRITINPDTSKNQFSLQLNSVTPEDTAVYYCARXWGQGTLVTVSSGSASAPTLFPLVSCENSPSDTSSVAVGCLAQDFLP

SEQ ID NO: 9 - protein, 91 aa, Homo sapiens
  MISC_FEATURE (1)..(91): Vh7 heavy chain variable region
QVQLVQSGSELKKPGASVKVSCKASGYTFTXWVRQAPGQGLEWMGXRFVFSLDTSVSTAYLQISSLKAEDTAVYYCARXWGQGTLVTVSSS

SEQ ID NO: 10 - protein, 93 aa, Homo sapiens
  MISC_FEATURE (1)..(93): Kappa1_4 light chain variable region
DIQMTQSPSSLSASVGDRRVTITCXWYQQKPGKAPKLLIYXGVPSRFSGSGSGTDFTLTISSLQPEDFATYYCXFGQGTKVEIKRTVAAPSVF

SEQ ID NO: 11 - protein, 92 aa, Homo sapiens
  MISC_FEATURE (1)..(92): Kappa2 light chain variable region
DIVMTQSPLSLPVTPGQPASISCXWYLQKPGQSPQLLIYXGVPDRFSGSGSGTDFTLKISRVEAEDVGVYYCXFGQGTKVEIKRTVAAPSVF

SEQ ID NO: 12 - protein, 91 aa, Homo sapiens
  MISC_FEATURE (1)..(91): Kappa3 light chain variable region
EIVLTQSPGTLSLSPGERATLSCXWYQQKPGQAPRLLIYXGIPDRFSGSGSGTDFTLTISRLEPEDFAVYYCXFGQGTKVEIKRTVAAPSV

SEQ ID NO: 13 - protein, 85 aa, Homo sapiens
  MISC_FEATURE (1)..(85): Kappa5 light chain variable region
ETTLTQSPAFMSATPGDKVNISCXWYQQKPGEAAIFIIQXGIPPRFSGSGYGTDFTLTINNIESEDAAYYFCXLRHFWPGDQAAG

SEQ ID NO: 14 - protein, 79 aa, Homo sapiens
  MISC_FEATURE (1)..(67): KappaNew1 light chain variable region
EIVMTQSPVNLSMSAGEXWYQQKPGQAPRLFIYXGISARFSGSGSGTDFTLTITSLQSEDFAVYYCXFGQGTKLDIKRT

SEQ ID NO: 15 - protein, 77 aa, Homo sapiens
  MISC_FEATURE (1)..(65): KappaNew2 light chain variable region
ELTQSPGTLSLSPGEXWYQHKPGQAPRLVIHXGISDRFSGSGSGTDFTLTITRLEPEDFALYYCXFGQGTKLDFKRT

SEQ ID NO: 16 - protein, 98 aa, Homo sapiens
  MISC_FEATURE (1)..(98): Lambda1a light chain variable region
QSVLTQPPSVSGAPGQRVTISCXWYQQLPGTAPKLLIYXGVPDRFSGSKSGTSASLAISGLQSEDEADYYCXFGGGTKLTVLGQPKAAPSVTLFPPSS

SEQ ID NO: 17 - protein, 99 aa, Homo sapiens
  MISC_FEATURE (1)..(99): Lambda1b light chain variable region
AQSVLTQPPSVSAAPGQKVTISCXWYQQLPGTAPKLLIYXGIPDRFSGSKSGTSATLGITGLQTGDEADYYCXFGGGTKLTVLGQPKAAPSVTLFPPSS

SEQ ID NO: 18 - protein, 99 aa, Homo sapiens
  MISC_FEATURE (1)..(72): Lambda2 light chain variable region
QSALTQPASVSGSPGQSITISCXWYQQHPGKAPKLMIYXGVSNRFSGSKSGNTASLTISGLQAEDEADYYCXFGGGTTKLTVLGQPKAAPSVTLFPPSS

SEQ ID NO: 19 - protein, 107 aa, Homo sapiens
  MISC_FEATURE (1)..(107): Lambda3a light chain variable region
SYELTQPPSVSVSPGQTARITCXWYQQKPGQAPVLVIYXGIPERFSGSSSGTTATLTISGVQAEDEADYYCXFGGGTKLTVLGQPKAAPSVTLFPPSSEELQANKAT

SEQ ID NO: 20 - protein, 93 aa, Homo sapiens
  MISC_FEATURE (1)..(93): Lambda3b light chain variable region
SYVLTQPPSVSVAPGQTARITCXWYQQKPGQAPVLVVYDXGIPERFSGSNSGNTATLTISRVEAGDEADYYCXFGGGTKLTVLGQPKAAPTVT

SEQ ID NO: 21 - protein, 98 aa, Homo sapiens
  MISC_FEATURE (1)..(98): Lambda3c light chain variable region
  MISC_FEATURE (40)..(71): framework 3
SYELTQPPSVSVSPGQTASITCXWYQQKPGQSPVLVIYXGIPERFSGSNSGNTATLTISGTQAMDEADYYCXFGGGTKLTVLGQPKAAPSRSLCPPPP

SEQ ID NO: 22 - protein, 98 aa, Homo sapiens
  MISC_FEATURE (1)..(98): Lambda3e light chain variable region
SSELTQDPAVSVALGQTVRITCXWYQQKPGQAPVLVIYXGIPDRFSGSSSGNTASLTITGAQAEDEADYYCXFGGGTKLTVLGQPKAAPSVTLFPPSS

SEQ ID NO: 23 - protein, 94 aa, Homo sapiens
  MISC_FEATURE (1)..(94): Lambda4a light chain variable region
QPVLTQSSSASASLGSSVKLTCXWHQQQPGKAPRYLMKXGVPDRFSGSSSGADRYLTISNLQSEDEADYYCXFGGGTKLTVLGQPKAAPSVTLF

SEQ ID NO: 24 - protein, 95 aa, Homo sapiens
  MISC_FEATURE (1)..(95): Lambda4b light chain variable region
QLVLTQSPSASASLGASVKLTCXWHQQQPEKGPRYLMKXGIPDRFSGSSSGAERYLTISSLQSEDEADYYCXFGGIGGGTKLTVLGQPKAAPSVS

SEQ ID NO: 25 - protein, 88 aa, Homo sapiens
  MISC_FEATURE (1)..(75): Lambda5 light chain variable region
QAVLTQPSSLSASPGASASLTCXWYQQKPGSPPQYLLRYXGVPSRFSGSKDASANAGILLISGLQSEDEADYYCXFGGGTKLTVLSQP

SEQ ID NO: 26 - protein, 101 aa, Homo sapiens
  MISC_FEATURE (1)..(101): Lambda6 light chain variable region
NFMLTQPHSVSESPGKTVTISCXWYQQRPGSAPTTVIYXGVPDRFSGSIDSSSNSASLTISGLKTEDEADYYCXFGGGTKLTVLGQPKAAPSVTLFPPSSS

SEQ ID NO: 27 - protein, 89 aa, Homo sapiens
  MISC_FEATURE (1)..(72): Lambda7 light chain variable region
QAVVTQEPSLTVSPGGTVTLTCXWFQQKPGQAPRALIYXWTPARFSGSLLGGKAALTLSGVQPEDEAEYYCXFGGGTKLTVLGQPKAAP

SEQ ID NO: 28 - protein, 89 aa, Homo sapiens
  MISC_FEATURE (1)..(89): Lambda8 light chain variable region
QTVVTQEPSFSVSPGGTVTLTCXWYQQTPGQAPRTLIYXGVPDRFSGSILGNKAALTITGAQADDESDYYCXFGGGTKLTVLGQPKAAP

SEQ ID NO: 29 - protein, 91 aa, Homo sapiens
  MISC_FEATURE (1)..(91): Lambda9 light chain variable region
QPVLTQPPSASASLGASVTLTCXWYQQRPGKGPRFVMRXGIPDRFSVLGSGLNRYLTIKNIQEEDESDYHCXFGGGTKLTVLGQPKAAPSV

SEQ ID NO: 30 - protein, 87 aa, Homo sapiens
  MISC_FEATURE (1)..(87): Lambda10 light chain variable region
QAGLTQPPSVSKGLRQTATLTCXWLQQHQGHPPKLLSYXGISERFSASRSGNTASLTITGLQPEDEADYYCXFGGGTKLTVLGQPKA

SEQ ID NO: 31 - protein, 354 aa, Homo sapiens
  MISC_FEATURE (1)..(354): IgA1 heavy chain constant region
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSZEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 32 - protein, 340 aa, Homo sapiens
  MISC_FEATURE (1)..(340): IgA2 heavy chain constant region
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 33 - protein, 384 aa, Homo sapiens
  MISC_FEATURE (1)..(384): IgD heavy chain constant region
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 34 - protein, 497 aa, Homo sapiens
  MISC_FEATURE (1)..(497): IgE heavy chain constant region
ASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPVGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKDVCVEEAEGEAPWTWTGLCIFAALFLLSVSYSAALTLLMVQRFLSATRQGRPQTSLDYTNVLQPHA

SEQ ID NO: 35 - protein, 339 aa, Homo sapiens
  MISC_FEATURE (1)..(339): IgG1 heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESBNGQPENNYKTTPPVLDSDGSFFLYSKLTVDKSRWQQGNVFSCSVMHEALHNHYTQKSLSLSPGKTHTCPPCP

SEQ ID NO: 36 - protein, 326 aa, Homo sapiens
  MISC_FEATURE (1)..(326): IgG2 heavy chain constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 37 - protein, 377 aa, Homo sapiens
  MISC_FEATURE (1)..(377): IgG3 heavy chain constant region
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 38 - protein, 327 aa, Homo sapiens
  MISC_FEATURE (1)..(327): IgG4 heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 476 aa, Homo sapiens
  MISC_FEATURE (1)..(476): IgM heavy chain constant region
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRSKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWQMQRGQPLSPEKYVTSAPMPEPQAPGRYFAHSILTVSEEEWNTGETYTCVVAHEALPNRVTERTVDKSTGKPTSADEEGFENLWATASTFIVLYNVSLVMSDTAGTCYVK

SEQ ID NO: 40 - protein, 107 aa, Homo sapiens [sequence omitted]

SEQ ID NO: 41 - protein, 107 aa, Homo sapiens
  MISC_FEATURE (1)..(107): Light chain lambda constant region (IgLambda)
GQPKAAPSVTLFPPSSEELQANKATLVCLISDFYPGAVTVAWKADSSPVKAGVETTTPSKQSNNKYAASSYLSLTPEQWKSHRKSYSCQVTHEGSTVEKTVAPTECS

(19) United States
(12) Patent Application Publication
(10) Pub. No.: US 2006/0088845 A1
(43) Pub. Date: Apr. 27, 2006

(54) Method and apparatus for analyzing and generating human antibody amino acid and nucleic acid sequences

(76) Inventor: Jin Lu, Boothwyn, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/091,234

(22) Filed: Mar. 28, 2005

Related U.S. Application Data
(60) Provisional application No. 60/558,090, filed on Mar. 31, 2004.

(51) Int. Cl. C12Q 1/68; G01N 33/53; G06F 19/00

(52) U.S. Cl. 435/6; 435/7.1; 702/19

(57) ABSTRACT

The invention provides methods, computer programs, data and databases, computer readable media, computer systems, and/or apparatus that use, compare or generate data corresponding to at least one partial antibody or antibody fusion protein nucleic acid or amino acid sequence, on recordable media or in computer memory, such as engineered antibody or antibody fusion protein sequences that include any combination of partial antibody sequences, as well as comparisons between different human antibody partial or full sequences, wherein the present invention can be used, inter alia, for research, diagnostic and/or therapeutic products, methods and devices.

### BACKGROUND OF THE INVENTION

This application claims priority to Provisional Application Ser. No. 60/558,090 filed Mar. 31, 2004, and is entirely incorporated herein by reference.

1. Field of the Invention

The present invention provides methods, computer programs, data and databases, computer readable media, computer systems, and/or apparatus that use, compare or generate data corresponding to at least one partial or complete antibody or antibody fusion protein nucleic acid or amino acid sequence, on recordable media or in computer memory, such as an antibody or antibody fusion protein sequences that include any combination of partial antibody sequences, as well as comparisons between different human antibody partial or full sequences, wherein the present invention can be used, inter alia, for research, diagnostic and/or therapeutic products, methods and devices.

2. Related Art

Since the initiation of genome sequencing projects, such as the Human Genome Project, there has been an explosion of amino acid and nucleic acid sequence information. Advancements in the areas of nucleic acid sequencing and protein sequencing have also played an important role in this information explosion. However, the development and refinement of tools to analyze these sequences has barely kept pace with the information explosion. At the same time, development of sophisticated techniques for producing monoclonal antibodies (MABs) with unique specificity have evolved.

MABs can function as research reagents, diagnostics or therapeutics. Antibody based therapeutics can potentially treat a broad spectrum of health threats such as autoimmune disorders, cancers, infections, or poisonings. However, non-human antibodies contain amino acid sequences that are immunogenic in humans. Consequently, it is desirable to employ fully human or humanized antibodies to limit the immunogenicity problems caused by immunogenic sequences in human patients.

Human antibody sequences can be analyzed to attempt to determine potential structural and functional information. Such information can provide insights into antibody structure, posttranslational modification, and expression. This information in turn can be used to rationally alter antibody half-life, affinity, expression, and even function. Such rational alterations can be accomplished by the deletion or substitution of single amino acid residues, or discrete regions, of an antibody.

To facilitate such rational approaches to antibody design it is necessary to have tools which enable the identification of conserved residues and regions of human antibodies. To meet this need there is a need for suitable methods, computer systems and networks, computer accessible databases, and/or algorithms.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention is directed to methods, computer programs, data, databases, computer readable media, computer systems, and/or apparatus for analyzing and generating human antibody sequences using novel approaches to analyze human antibody sequences and categorize classes, subclasses and components thereof, in order to provide searchable, analyzable and exportable databases and fields of amino acid and nucleic acid sequence data, as well as generating amino acid and nucleic acid sequence suitable to use in therapeutic and/or diagnostic antibodies, antibody fusion proteins or other protein sequences.

The present invention provides methods, computer programs, data and databases, computer readable media, computer systems, and/or apparatus that use, compare or generate data corresponding to at least one partial antibody or antibody fusion protein nucleic acid or amino acid sequence, on recordable media or in computer memory, such as engineered antibody or antibody fusion protein sequences that include any combination of partial antibody sequences, as well as comparisons between different human antibody partial or full sequences.

In one aspect of the present invention, a computer accessible database containing amino acid and/or nucleic acid sequences for consensus or engineered human antibodies or portions thereof is provided. The data in the database can optionally be processed and/or generated to filter out short and redundant sequences. In one embodiment of the database or data, there are provided at least one set of amino acid or nucleic acid sequences corresponding to and comprising at least one set of human or human derived complementarity determing regions (CDRs), human heavy or light chain variable and/or constant region sequences, and/or human or human derived constant region sequences in the database. The data, in this non-limiting example of a database of the invention, can optionally be organized by grouping, superfamily, family and/or subfamily. Multiple data displays can optionally be available for analyzing, generating or viewing data in the database.

In a further aspect of the present invention, a BLAST or similar search engine is optionally further provided for searching, analyzing or generating at least one part of the database (see, e.g., as known in the art, e.g., but not limited to as disclosed in, Altschul et al., Nucleic Acids Res. 25:3389-3402 (1997), entirely incorporated herein by reference).

In another aspect, the present invention provides at least one algorithm for generating at least one set of clustered alignments of human antibody amino acid or nucleic sequences. In one embodiment, an algorithm classifies the collected constant and/or variable region sequence data into superfamilies, families, and/or subfamilies. The classifications can optionally be based on annotations and sequence similarity.

In yet another aspect of the present invention, an additional algorithm displaying the frequency of substitutions at each position in the clustered alignment is provided. In one embodiment, an algorithm determines the prototypical sequence for a given subfamily and the frequency of each substitution (amino acid residue or gap) occurring at the prototype position.

In one aspect of the invention, a method for comparing, analyzing and/or generating human antibody amino acid and/or nucleic acid sequences is provided. The method comprises at least one of the following steps, such as, but not limited to, at least one of:

101. accessing suitable antibody sequence databases and collecting constant, complimentarity determing regions (CDRs), and/or variable region sequences;

102. subjecting the data collected in step 101 to Algorithm 1, wherein the sequences are classified into groups, superfamilies, and/or subfamilies;

103. performing sequence alignment on all sequences assigned to a given subfamily in step 102;

104. displaying subfamily multiple sequence alignment result of step 103;

105. accessing antibody sequence databases and collecting variable region sequences;

106. subjecting the data collected in step 105 to Algorithm 2, wherein the variable region sequences are classified into superfamilies and subfamilies;

107. performing multiple sequence alignment on all sequences assigned to a given subfamily in step 106;

108. displaying subfamily multiple sequence alignment result of step 107;

109. subjecting the multiple sequence alignment data generated in step 103 or 107 to Algorithm 3, wherein each amino acid substitution is examined and the substitution's frequency of occurrence at a given position is calculated;

110. determining the constant region subfamily prototype sequence and substitutions;

111. displaying the the constant region subfamily prototype sequence and substitutions generated by step 110;

112. determining the variable region subfamily prototype sequence and substitutions;

113. displaying the variable region subfamily prototype sequence and substitutions generated by step 112;

114. exporting the displayed results from step 104, 108, 111 or 113 to a web interface, wherein the displays can be viewed and BLAST searching can be performed.

In another aspect, the present invention provides a computer accessible database of clustered alignments of all human antibody amino acid sequences. In one non-limiting embodiment, the heavy chain variable region antibody superfamily consists of a total of 6628 unique sequences belonging to 9 subfamilies. In another embodiment, the light chain variable region kappa superfamily consists of 1730 unique sequences belonging to 6 subfamilies. In yet another embodiment, the light chain variable region lambda superfamily consists of 1209 unique sequences belonging to 15 subfamilies. In still another embodiment, there are 92 unique human constant region sequences belonging to 7 superfamilies and among them, IgA heavy chain constant region superfamily contains 2 subfamilies and the IgG heavy chain constant region superfamily contains 4 subfamilies.

In still a further aspect of the present invention, a computer program product is provided that has computer program logic recorded thereon for enabling a processor in a computer system to analyze and generate human antibody nucleic acid or amino acid sequences. Such computer program logic includes at least one of the following:

at least one algorithm, sub-routine, routine or means for enabling the processor to access antibody sequence databases and collect human antibody constant region sequences, wherein the sequences are classified into superfamilies and subfamilies;

at least one algorithm, sub-routine, routine or means for enabling the processor to access public available databases and collect human antibody variable region sequences, wherein the sequences are classified into superfamilies and subfamilies; and

at least one algorithm, sub-routine, routine or means for enabling the processor to determine the prototypical sequence for a subfamily and the frequency of each amino acid substitution occurring at the prototype position.

In still a further aspect of the present invention, the present invention provides a computer network, wherein the computer accessible databases, algorithms and computerized search system of the invention are assembled and operated on. The computer network comprises a browser or workstation connected via a first network to a server. This first network can be connected via a second network to additional browsers or workstations.

### FEATURES AND ADVANTAGES

The present invention provides methods, computer programs, data and databases, computer readable media, computer systems, and/or apparatus that use, compare or generate data corresponding to at least one partial antibody or antibody fusion protein nucleic acid or amino acid sequence, on recordable media or in computer memory, such as engineered antibody or antibody fusion protein sequences that include any combination of partial antibody sequences, as well as comparisons between different human antibody partial or full sequences, wherein the present invention can be used, inter alia, for research, diagnostic and/or therapeutic products, methods and devices.

### BRIEF DESCRIPTION OF THE DRAWINGS

The present invention is described with reference to the accompanying drawings. In the drawings, like reference numbers indicate identical or functionally similar elements.

FIG. 1 is a block diagram illustrating the overview of inputs, database assembly and analysis outputs in accordance with one embodiment of the present invention;

FIG. 2 is a block diagram illustrating Algorithm 1 analysis of constant region sequences in accordance with one embodiment for carrying out steps 101-104 shown in FIG. 1;

FIG. 3 is a block diagram illustrating Algorithm 2 analysis of variable region sequences in accordance with one embodiment for carrying out steps 105-108 shown in FIG. 1;

FIG. 4 is a block diagram illustrating Algorithm 3 for statistics distribution in accordance with one embodiment for carrying out steps 109-111 shown in FIG. 1;

FIG. 5 is a block diagram illustrating an exemplary computer system suitable for use with the present invention;

FIG. 6 is a screen shot (HTML page) depicting the content page with data display section hyperlinks exemplified using the heavy chain variable region Vh9 subfamily;

FIG. 7 is a screen shot depicting the first data display section wherein the raw optimized multiple sequence alignment with annotations of functional units generated by Algorithm 1 or 2 was exemplified using 25 Vh9 subfamily members;

FIG. 8 is a screen shot depicting the second data display section wherein the graphic alignment generated by Algorithm 1 or 2 was exemplified using 25 Vh9 subfamily members;

FIG. 9 is a screen shot depicting the third data display section generated by Algorithm 3 wherein the calculated amino acid distribution statistics for each prototype position distribution was exemplified using Vh9 subfamily sequence alignment, wherein the display is formatted for data export via a cut and paste function to programs such as Excel or Vector NTI; and

FIG. 10 is a screen shot depicting the fourth data display section presenting the same contents as the third data display in FIG. 9, wherein the display is designed for easy web page display on a computer monitor and for printing.

### DETAILED DESCRIPTION OF THE EMBODIMENTS

The present invention provides methods, computer programs, data and databases, computer readable media, computer systems, and/or apparatus that use, compare or generate data corresponding to at least one partial antibody or antibody fusion protein nucleic acid or amino acid sequence, on recordable media or in computer memory, such as engineered antibody or antibody fusion protein sequences that include any combination of partial antibody sequences, as well as comparisons between different human antibody partial or full sequences, wherein the present invention can be used, inter alia, for research, diagnostic and/or therapeutic products, methods and devices.

### Definitions

“Bad clusterings” are clusters which are inconsistent with the subfamily clusters of a “reference database” or a “classification database.”

A “browser” is a computer running a computer program for collecting and displaying accessible data.

“Calculated frequency” means the number of times a prototype residue occurs per the total number of sequences in the alignment inputted into Algorithm 3.

A “classification database” contains “reference sequences” which have been classified based on their germline subfamily. An example of a “classification database” is V Base (www.mrc-cpe.cam.ac.uk/vbase-ok.php?menu=901) which provides, when available, the germline subfamily of each variable heavy, variable light chain kappa, or variable light chain lambda included in the database.

A “cluster” is an organizational unit of sequences, or other string of characters, related by a given stringency. Clusters will vary depending on the chosen stringency.

A “duplicate sequence” in a collection of sequences is a sequence which is identical to at least one other sequence in the population.

“Gap frequency” is the percentage of all substitutions in a position which are gaps.

“Good clusterings” are consistent with the subfamily clusters of a “classification database.”

“Known subfamily annotations” are annotations indicating the subfamily of a sequence.

“Known superfamily annotations” are annotations which indicate the superfamily of a sequence.

A “network” is an interconnected or interrelated chain, group, or system such as for example a system of computers connected by communications lines.

A “prototype residue” is the amino acid residue which occurs most frequently in a single position of a multiple sequence alignment.

A “prototype sequence” corresponds to a sequence of “prototype residues.”

A “reference database” may be used to obtain heavy or light chain constant region “reference sequences.” Swissprot is one example of such a database, but other databases in which the subfamily of the sequences deposited in the database is indicated can function as a “reference database.”

“Reference sequences” are sequences which are known to belong to a given subfamily.

A “server” is a computer in a network which provides services to other computers in a network. Services provided by a server may include, for example, access to a database, files, and shared peripherals or the routing of files. A server may provide access to a database or files via a web server which provides such access to a “browser.”

“Short sequences” are 70 amino acid residues or less.

“Substitutions” may be amino acid residues or gaps (no residue) occurring at a position in an aligned set of sequences.

A “workstation” is a computer for running the algorithms of the invention, assembling the database of the invention or for software development. A “workstation” is also capable of displaying data or operating as a “browser.”

### Network Structure

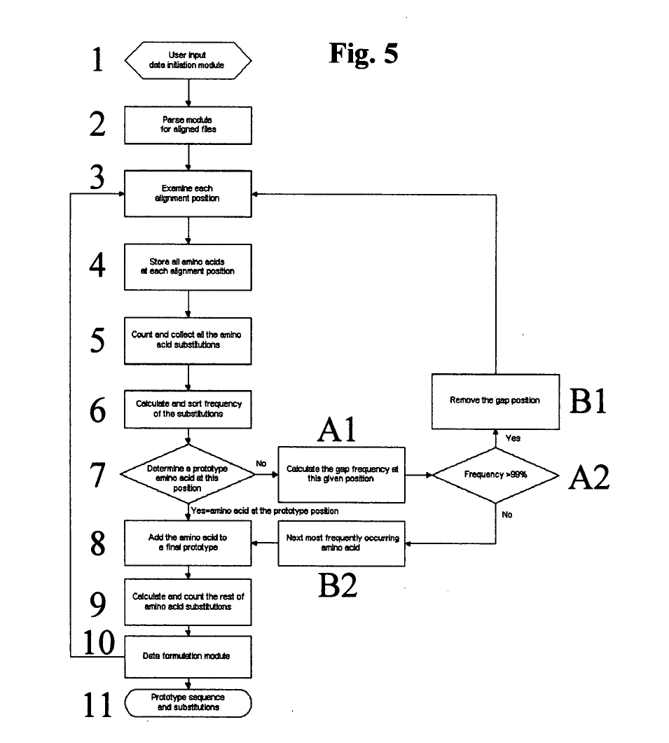

The computer accessible database, algorithms and computerized search system of the invention are assembled and operated on a computer network (FIG. 5). The computer network comprises a browser or workstation connected via a first network or a direct connection to a server. This first network can be connected via a second network to additional browsers or workstations.

### Database Contents and Features

The computer accessible database of the invention contains publicly available amino acid sequences for human antibodies. The data in the database is processed to filter out short and redundant sequences.

In one embodiment, there are 6628 unique human heavy chain variable region sequences, 1730 unique human light chain kappa variable region sequences, 1209 unique human light chain lambda variable region sequences, and 92 unique human constant region sequences in the database. The data in the database is organized by superfamily, and subfamily. Multiple data displays are available for viewing data in the database.

A BLAST or similar search engine is also provided for searching the database (e.g., as described in Altschul et al., Nucleic Acids Res. 25:3389-3402 (1997), and/or as known in the art).

### Superfamilies

The human antibody database, as a non-limiting exemplary embodiment, n contains non-redundant data, classified into 10 superfamilies (3 variable region sequence families and 7. constant region sequence families). The 3 variable region superfamilies are the heavy chain Vh, light chain V kappa and light chain V lambda superfamilies. The 7 constant region superfamilies are the heavy chain IgA, IgG, IgD, IgE, IgM, light chain constant kappa and light chain constant lambda superfamilies. Each superfamily, is further classified into at least one subfamilies (Table 1).

TABLE 1
Non-Limiting example of consensus antibody amino sequences according to the present invention

| SEQ ID | | Ab | | regions | | | | | | |
|---|---|---|---|---|---|---|---|---|---|---|
| NO | | region | AA NO | FR1 | CDR1 | FR2 | CDR2 | FR3 | CDR3 | FR4 |
| 1 | heavy | Vh1 | 125 | 1-31 | 32 | 33-46 | 47 | 48-79 | 80 | 81-125 |
| 2 | chain | Vh2 | 97 | 1-30 | 31 | 32-45 | 46 | 47-78 | 79 | 80-97 |
| 3 | variable | Vh3a | 102 | 1-30 | 31 | 32-45 | 46 | 47-78 | 79 | 80-102 |
| 4 | region | Vh3b | 102 | 1-30 | 31 | 32-45 | 46 | 47-78 | 79 | 80-102 |
| 5 | | Vh3c | 94 | 1-30 | 31 | 32-45 | 46 | 47-78 | 79 | 80-94 |
| 6 | | Vh4 | 106 | 1-30 | 31 | 32-45 | 46 | 47-78 | 79 | 80-106 |
| 7 | | Vh5 | 97 | 1-30 | 31 | 32-45 | 46 | 47-78 | 79 | 80-97 |
| 8 | | Vh6 | 91 | 1-30 | 31 | 32-45 | 46 | 47-78 | 79 | 80-91 |
| 9 | | Vh7 | 91 | 1-30 | 31 | 32-45 | 46 | 47-78 | 79 | 80-91 |
| 10 | light | κ1_4 | 73 | 1-23 | 24 | 25-39 | 40 | 41-72 | 73 | |
| 11 | chain | κ2 | 73 | 1-23 | 24 | 25-39 | 40 | 41-72 | 73 | |
| 12 | variable | κ3 | 73 | 1-23 | 24 | 25-39 | 40 | 41-72 | 73 | |
| 13 | region | κ5 | 73 | 1-23 | 24 | 25-39 | 40 | 41-72 | 73 | |
| 14 | | κ νεω1 | 67 | 1-17 | 18 | 19-33 | 34 | 35-66 | 67 | |
| 15 | | κ νεω2 | 65 | 1-15 | 16 | 17-31 | 32 | 33-64 | 65 | |
| 16 | | λ1α | 72 | 1-22 | 23 | 24-38 | 39 | 40-71 | 72 | |
| 17 | | λ1β | 73 | 1-23 | 24 | 25-39 | 40 | 41-72 | 73 | |
| 18 | | λ1_{χ} | 72 | 1-22 | 23 | 24-38 | 39 | 40-71 | 72 | |
| 19 | | λ3α | 72 | 1-22 | 23 | 24-38 | 39 | 40-71 | 72 | |
| 20 | | λ3β | 72 | 1-22 | 23 | 24-38 | 39 | 40-71 | 72 | |
| 21 | | λ3_{χ} | 72 | 1-22 | 23 | 24-38 | 39 | 40-71 | 72 | |
| 22 | | λ3ε | 72 | 1-22 | 23 | 24-38 | 39 | 40-71 | 72 | |
| 23 | | λ4α | 72 | 1-22 | 23 | 24-38 | 39 | 40-71 | 72 | |
| 24 | | λ4β | 72 | 1-22 | 23 | 24-38 | 39 | 40-71 | 72 | |
| 25 | | λ5 | 75 | 1-22 | 23 | 24-39 | 40 | 41-74 | 75 | |
| 26 | | λ6 | 74 | 1-22 | 23 | 24-38 | 39 | 40-73 | 74 | |
| 27 | | λ7 | 72 | 1-22 | 23 | 24-38 | 39 | 40-71 | 72 | |
| 28 | | λ8 | 72 | 1-22 | 23 | 24-38 | 39 | 40-71 | 72 | |
| 29 | | λ9 | 72 | 1-22 | 23 | 24-38 | 39 | 40-71 | 72 | |
| 30 | | λ10 | 72 | 1-22 | 23 | 24-38 | 39 | 40-71 | 72 | |

| SEQ ID | | | TOTAL | regions | | | | | | |
|---|---|---|---|---|---|---|---|---|---|---|
| NO | | | AA NO | CH1 | hinge1 | hinge2 | hinge3 | hinge4 | CH2 | CH3 |
| 31 | heavy | IgA1 | 354 | 1-102 | 103-122 | | | | 123-222 | 223-354 |
| 32 | chain | IgA2 | 340 | 1-102 | 103-108 | | | | 109-209 | 210-340 |
| 33 | constant | IgD | 384 | 1-101 | 102-135 | 136-159 | | | 160-267 | 268-384 |
| 34 | region | IgE | 497 | 1-103 | | | | | 104-210 | 211-318 |
| 35 | | IgG1 | 339 | 1-98 | 99-113 | | | | 114-223 | 224-339 |
| 36 | | IgG2 | 326 | 1-98 | 99-110 | | | | 111-219 | 220-326 |
| 37 | | IgG3 | 377 | 1-98 | 99-115 | 116-130 | 131-145 | 146-160 | 161-270 | 271-377 |
| 38 | | IgG4 | 327 | 1-98 | 99-110 | | | | 111-220 | 221-327 |
| 39 | | IgM | 476 | 1-104 | | | | | 105-217 | 218-323 |
| 40 | light | Igkc | 107 | | | | | | | |
| 41 | chain | Igl | 107 | | | | | | | |
| | constant | | | | | | | | | |
| | region | | | | | | | | | |

### Subfamilies

Subfamilies are sorted and displayed from largest to smallest based on the number of sequences in each subfamily. The heavy chain variable region antibody superfamily consists of a total of 6628 unique sequences belonging to 9 subfamilies. The light chain variable region kappa and light chain variable region lambda superfamilies contain 6 and 15 subfamilies respectively. The light chain variable region kappa superfamily has 1730 sequences; the light chain variable region lambda superfamily contains 1209 sequences. The heavy chain constant region superfamilies consist of IgA, IgG, IgD, IgE, and IgM. The IgA heavy chain constant region superfamily contains two subfamilies and the IgG heavy chain constant region superfamily has 4 subfamilies. The other heavy chain constant region superfamilies each contain a single subfamily. The total number of unique heavy chain constant regions sequences in the database is 92. The light chain kappa and lambda superfamilies each contain a single subfamily.

### Algorithms

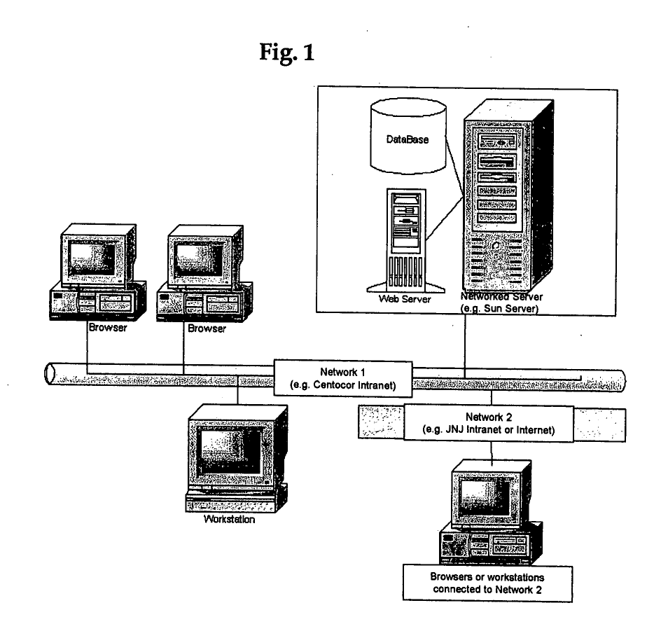

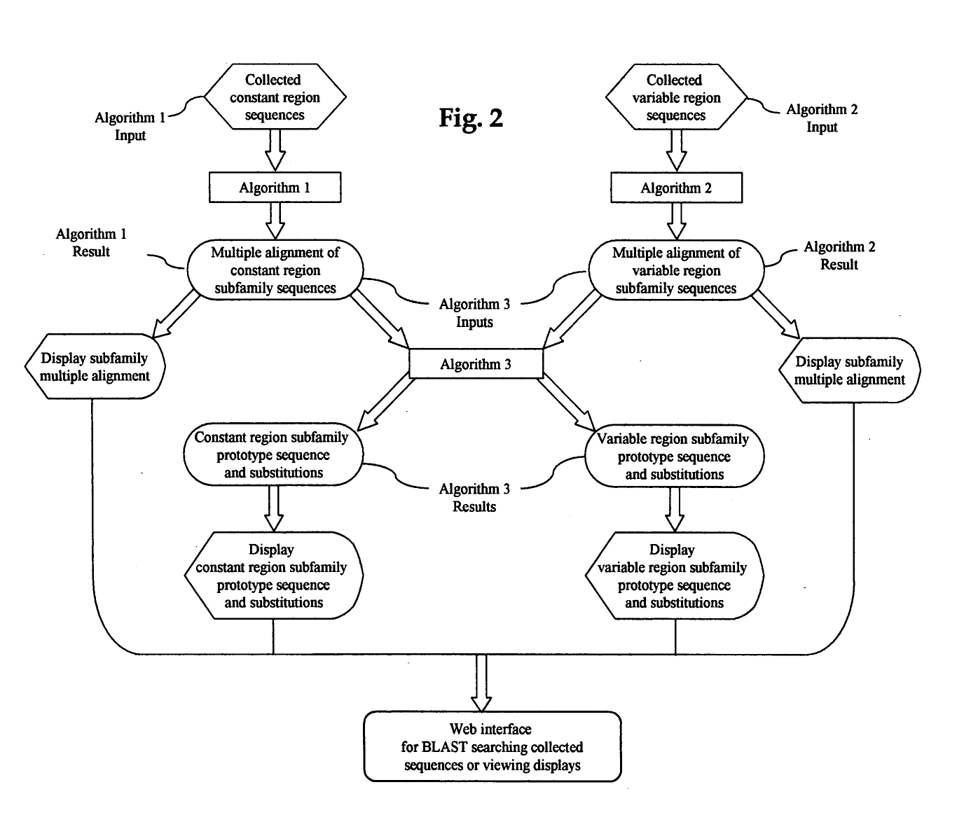

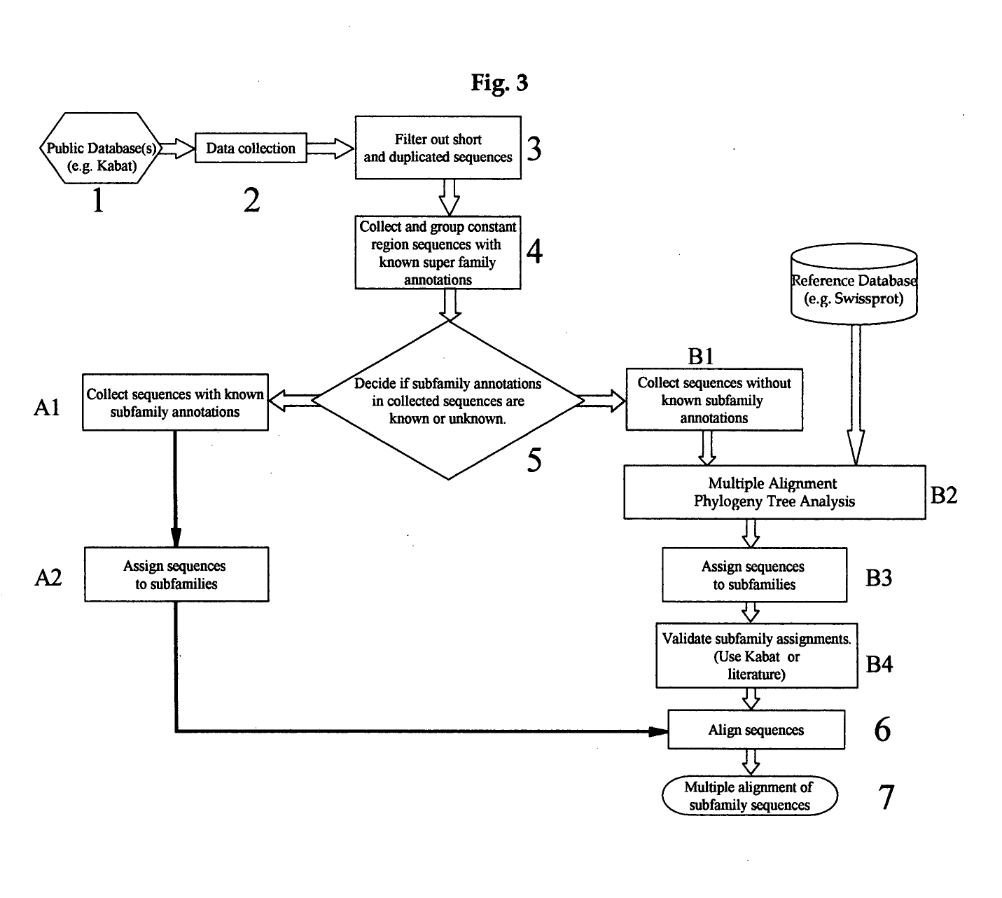

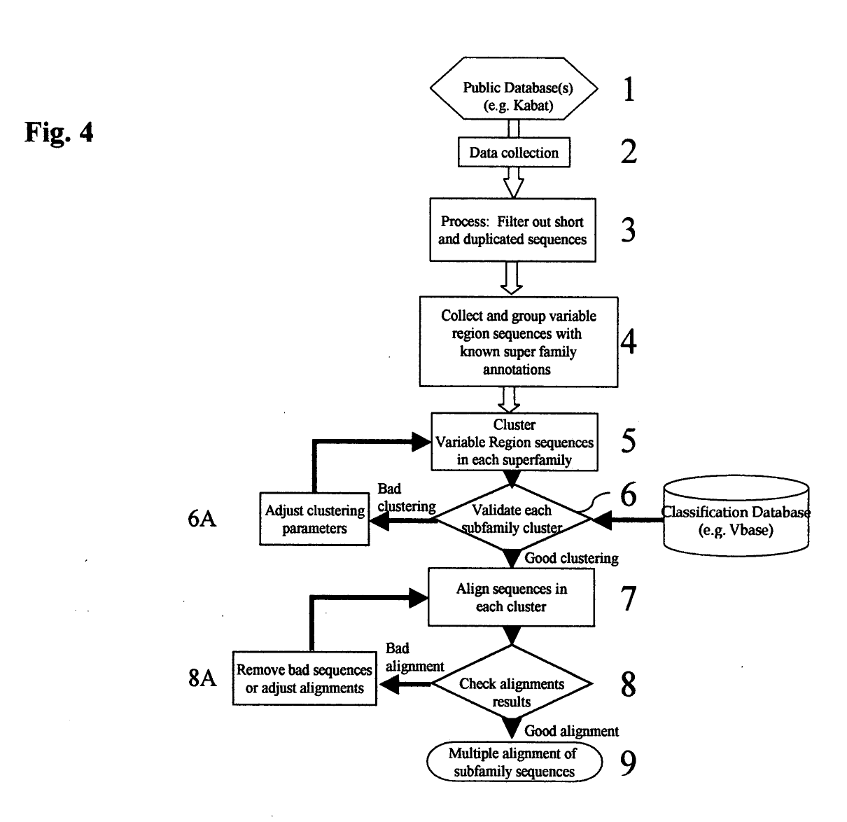

Two algorithms (FIGS. 2 and 3) classify the collected data in the database into 10 different superfamilies (3 for variable region sequences, 7 for constant region sequences) and into corresponding subfamilies based on annotations and sequence similarity. A third algorithm (FIG. 4) determines the prototypical sequence for a subfamily and the frequency of each substitution (amino acid residue or gap) occurring at the prototype position. All three algorithms can be used to construct the database of the invention or a portion thereof (overviewed in FIG. 1).

### Algorithm 1: Generation of Constant Region Multiple Sequence Alignments and Subfamily Assignments

The first algorithm (FIG. 2) collects, classifies, and analyzes the heavy and light chain constant regions of human antibodies.

First, a preparation step is performed in which human antibody databases such as Kabat (immuno.bme.nwu.edu; available via NCBI), NCBI (www.ncbi.nlm.nih.gov), SwissProt (www.ebi.ac.uk/swissprot/; available via NCBI), PIR (pir.georgetown.edu), published patent sequence databases (e.g. PAT database; available via NCBI) or other publicly available data sources are made accessible to a workstation for further analysis. See FIG. 2, Step 201.

Second, a process step is performed in which data for human light and heavy chain constant region amino acid sequences are collected. In this step, human light and heavy chain constant region sequences are collected based on annotations indicating the sequence is an antibody or immunoglobulin, that the sequences is a Homo sapiens (human) sequence, and that the sequences is a heavy or light chain constant region sequence. Data collected in this step includes annotations, sequences, sequence names, accession numbers and the like. See FIG. 2, Step 202.

Third, a process step is performed in which duplicate and short sequences are removed from the collected data. See FIG. 2, Step 203.

Fourth, a process step is performed in which data for light and heavy chain constant region sequences with known superfamily annotations is collected and grouped by superfamily. In the context of this step known superfamily annotations indicate that a sequence is a heavy chain constant IgA, IgD, IgE, IgG or IgM superfamily member or alternatively that the sequence is a light chain constant lambda or kappa superfamily member. Sequences lacking known superfamily annotations are not collected at this step. See FIG. 2, Step 204.

Fifth, a decision step is performed in which it is determined if a sequence has known subfamily annotations. See FIG. 2, Step 205.

The fifth step is a branch point. After this step sequences with known subfamily annotations are assigned to subfamilies through different steps than those without known subfamily annotations. Each branch (Branch A and Branch B) is described below. After the sequences have been assigned to subfamilies they are further processes in a sixth step.

The sixth step, is process step in which a multiple sequence alignment is performed on all sequences assigned to a given subfamily. A program such as, for example, CLUSTALW (Higgins et al., Nucleic Acids Res. 22:4673-4680 (1994)) may be used to perform multiple sequence alignments. See FIG. 2, Step 206.

The seventh step is a terminal step in which the result of the preceding steps, a multiple sequence alignment of all the sequences in a given constant region subfamily, may either be displayed or input into Algorithm 3. Data may be displayed in the first section data display described below. See FIG. 2, Step 207.

Branch A: Processing of Sequences with Known Subfamily Annotations

First, a process step is performed in which sequences with known subfamily annotations are collected. See FIG. 2, Step 205A1.

Second, a process step is performed in which these sequences are assigned to subfamilies. See FIG. 2, Step 205A2.

After these steps the sequences with known subfamily annotations are processed identically to sequences without known subfamily annotations. Identical processing resumes at step 6. See FIG. 2, Step 206.

Branch B: Processing of Sequences without Known Subfamily Annotations

First, a process step is performed in which sequences without known subfamily annotations are collected. See FIG. 2, Step 205B1.

Second, a process step is performed in which multiple sequence alignment and phylogeny tree analysis is performed to generate clusters of sequences. A program such as, for example, CLUSTALW may be used to perform multiple sequence alignments. The sequences processed in this step include both collected sequences without known subfamily annotations and reference sequences from a reference database. The reference sequences have known subfamily annotations such as for example IgG1, IgG2, IgA1, IgA2 and the like. Including reference sequences in this step provides a tag which can be used to determine which subfamily each cluster generated by the multiple alignment and phylogeny tree analysis corresponds to. See FIG. 2, Step 205B2.

Third, a process step is performed in which the sequences are assigned to subfamilies based on which reference sequences cluster with them in the phylogeny tree. See FIG. 2, Step 205B3.

Fourth, a process step is performed in which the subfamily assignments are validated by user examination of the scientific literature. Subfamily assigments are validated if they are consistent with subfamily assignments described in the scientific literature. See FIG. 2, Step 205B4.

After these steps the sequences without known subfamily annotations are processed identically to sequences with known subfamiliy annotations. Identical processing resumes at step 206. See FIG. 2, Step 206.

### Algorithm 2: Generation of Variable Region Multiple Sequence Alignments and Subfamily Assignments

The second algorithm (FIG. 3) collects, classifies, and analyzes the heavy and light chain variable regions of human antibodies.

First, a preparation step is performed in which human antibody databases such as Kabat (immuno.bme.nwu.edu; available via NCBI), NCBI (www.ncbi.nlm.nih.gov), SwissProt (www.ebi.ac.uk/swissprot/; available via NCBI), PIR (pir.georgetown.edu), published patent sequence databases (e.g. PAT database; available via NCBI) or other publicly available data sources are made accessible to a workstation for further analysis. See FIG. 3, Step 301.

Second, a process step is performed in which data for human light and heavy chain variable region amino acid sequences is collected. In this step, human light and heavy chain variable region sequences are collected based on annotations indicating the sequence is an antibody or immunoglobulin, that the sequences is a Homo sapiens (human) sequence, and that the sequences is a heavy or light chain variable region sequence. Data collected in this step includes annotations, sequences, sequence names, accession numbers and the like. See FIG. 3, Step 302.

Third, a process step is performed in which duplicate and short sequences are removed from the collected data. See FIG. 3, Step 303.

Fourth, a process step is performed in which data for light and heavy chain variable region sequences with known superfamily annotations is collected and grouped by superfamily. In the context of this step known superfamily annotations indicate that a sequence is a heavy chain variable region, light chain lambda variable region, or light chain kappa variable region superfamily member. Sequences lacking known superfamily annotations are not collected at this step. See FIG. 3, Step 304.

Fifth, a process step is performed in which sequences within each superfamily are clustered to identify the corresponding subfamilies. This clustering is based on sequence similarity and is performed using a single linkage clustering algorithm (e.g. the BlastClust program; ftp://ftp.ncbi.nih.gov/blast/execu tables). See FIG. 3, Step 305.

Sixth, a decision step is performed in which the subfamily clusters are compared to the germline subfamilies of a classification database (e.g. V Base antibody database) and it is decided if the clustering is a good clustering or bad clustering. This comparison is possible because variable region reference sequences from each germline subfamily found in the classification database are present among the variable region sequences which have been collected and clustered. See FIG. 3, Step 306.

One example of good clustering, in the context of the sixth step, occurs when each cluster of collected sequences contains reference sequences belonging solely to a single germline subfamily of the classification reference. Those of ordinary skill in the art will also recognize other examples of good clustering.

One example of bad clustering, in the context of the sixth step, occurs when a single cluster of collected sequences contains reference sequences belonging to several different germline subfamilies. Those of ordinary skill in the art will also recognize other examples of bad clustering.

If bad clustering is detected a process step is performed in which the clustering parameters (e.g. overlap, percent sequence identity and the like) of the single linkage clustering algorithm are adjusted. The clustering and validation steps are then repeated until a good cluster is obtained. See FIG. 3, Step 306A.

The seventh step is performed when good subfamily clustering is obtained. This step is a process step in which a multiple sequence alignment of the sequences in each subfamily cluster is performed. A program such as, for example, CLUSTALW may be used to perform multiple sequence alignments. See FIG. 3, Step 307.

Eighth, a decision step is performed in which these alignments are determined to be good or bad. Bad or good alignments may be recognized by those skilled in the art by examination of a given alignment. See FIG. 3, Step 308.

If the alignment is bad it is improved by removing sequences or adjusting the alignment. See FIG. 3, Step 308A.

The ninth step is performed, when a good alignment is obtained. The ninth step is a terminal step in which the result of the preceding steps, a multiple sequence alignment of all the sequences in a given variable region subfamily, may either be displayed or input into Algorithm 3. Data may be displayed in the first section data display described below. See FIG. 3, Step 309.

### Algorithm 3: Generation of Prototype Amino Acid Sequences and Calculation of Substitution Frequency

A third algorithm (FIG. 4) reports each amino acid substitution and the substitution's frequency of occurrence at a given position in a subfamily's prototype sequence.

First, a preparation step is performed in which multiple sequence alignment and data formatting instructions are inputted by a user into the data initiation module. See FIG. 4, Step 401.

The inputted multiple sequence alignment data may be generated by Algorithm 1 or Algorithm 2. See FIG. 2 or 3. Such data includes multiple sequence alignments corresponding to a variable or constant region subfamily. The data formatting instructions specify the number of amino acid prototype positions, substitutions and other information to display per row. This information is used by the data formulation module described below in step 410. See FIG. 4, Step 410.

Second, a process step is performed in which the multiple sequence alignment data is parsed to collect the substitutions occurring at all positions in a set of aligned sequences. See FIG. 4, Step 402.

Third, a process step is performed in which a single position in the multiple sequence alignment is examined. See FIG. 4, Step 403.

Fourth, a process step is performed in which each substitution occurring at a single examined position in a set of aligned sequences is collected. See FIG. 4, Step 404.

Fifth, a process step is performed in which all the substitutions occurring at a single position in a set of aligned sequences are counted and collected. See FIG. 4, Step 405.

Sixth, a process step is performed in which a calculation of the frequency of each substitution is made and the substitutions are sorted. Substitutions are sorted from most common to least common based on the number of times the substitution occurs in a single position. See FIG. 4, Step 406.

Seventh, a decision step is performed in which it is determined if an amino acid residue is the most frequent substitution occurring in a position. If an amino acid residue is the most frequently occurring substitution the decision is to proceed to step 8.

See FIG. 4, Step 407.

If a gap is the most frequently occurring substitution the decision is to proceed to step 407A1 described below. Step 407A1 is a branch for the processing of positions in which the most frequent substitution is a gap. See FIG. 4, Step 407A1.

Eighth, a process step is performed in which the most frequently occurring amino acid residue in a position is designated to be the prototype residue for the position. See FIG. 4, Step 408.

Ninth, a process step is performed in which a count is made of the number of times each substitution occurs in a position and the calculated frequency for the position's prototype residue is generated. See FIG. 4, Step 409.

Tenth, a process step is performed by the data formulation module in which the preceding steps are repeated via a do-loop for each position in the inputted multiple sequence alignment. After these steps have been performed for each position in the alignment the module reads the formatting instructions inputted by the user into the data initiation module of step 401. The data is then formatted for display. See FIG. 4, Step 410.

The eleventh step is a terminal step in which the result of the preceding steps, a prototype sequence and substitutions for each position of this sequence, may be displayed. Data may be displayed in the third or fourth section data displays described below. See FIG. 4, Step 411.

Branch A: Processing of Positions in which the Most Frequent Substitution is a Gap

First, a process step is performed in which a calculation is made of the gap frequency. See FIG. 4, Step 407A1.

Second, a decision step is performed in which it is determined if the gap frequency is more or less than 99 percent. See FIG. 4, Step 407A2.

If the gap frequency is more than 99 percent the position is removed from the dataset and steps three through seven are repeated. See FIG. 4, Step 407B1.

If the gap frequency is less than 99 percent the most frequent amino acid residue is identified and step eight is performed. See FIG. 4, Step 407B2.

### Database Organization and Data Displays

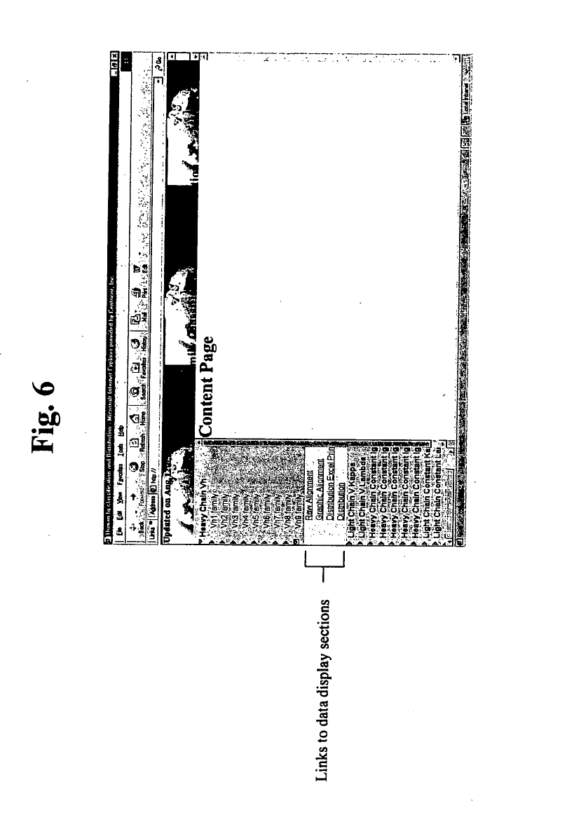

For each subfamily in a given superfamily there are four data display sections (FIG. 6).

First Section Data Display

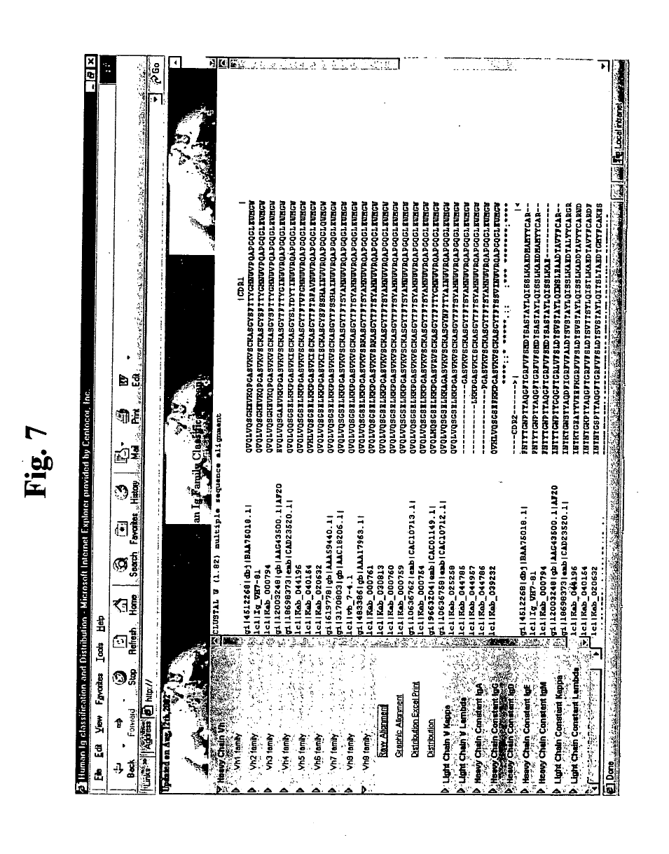

In the first section data display there is an optimized multiple sequence alignment which consists of all sequences in a subfamily and includes annotations of functional units such as frameworks, CDRs, CH1-4, or hinge regions (Kabat et al., 1991) (FIG. 7). The source of each sequence can be discerned through their names, e.g. lcl|Kab_{—}000794 which indicates the sequence is from the Kabat database (Johnson, G. et al., 2000) (FIG. 7). The data for the first section displays can be generated via algorithm 1 or 2 as appropriate.

Second Section Data Display

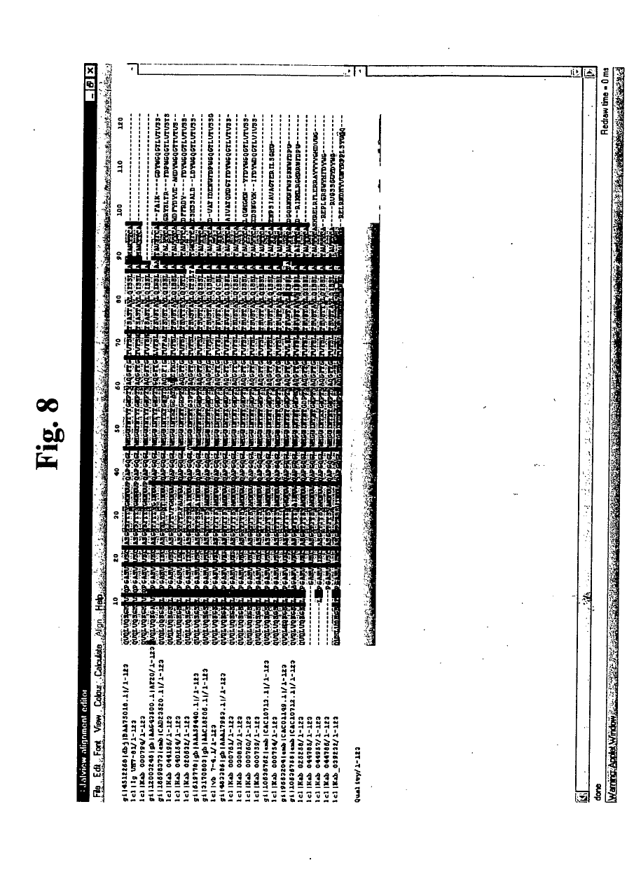

In the second section data display, which is a graphic alignment, each amino acid is color coded according to its charge, hydrophobicity or other properties (FIG. 8). The conserved regions and broad pattern for alignments can easily be observed in the second section display. The data for the second section data displays can be generated by algorithm 1 or 2 as appropriate. A program such as, for example, JALVIEW (http://www.ebi.ac.uk/˜michele/jalview/dist/) may be used to generate a second section data display.

Third Section Data Display

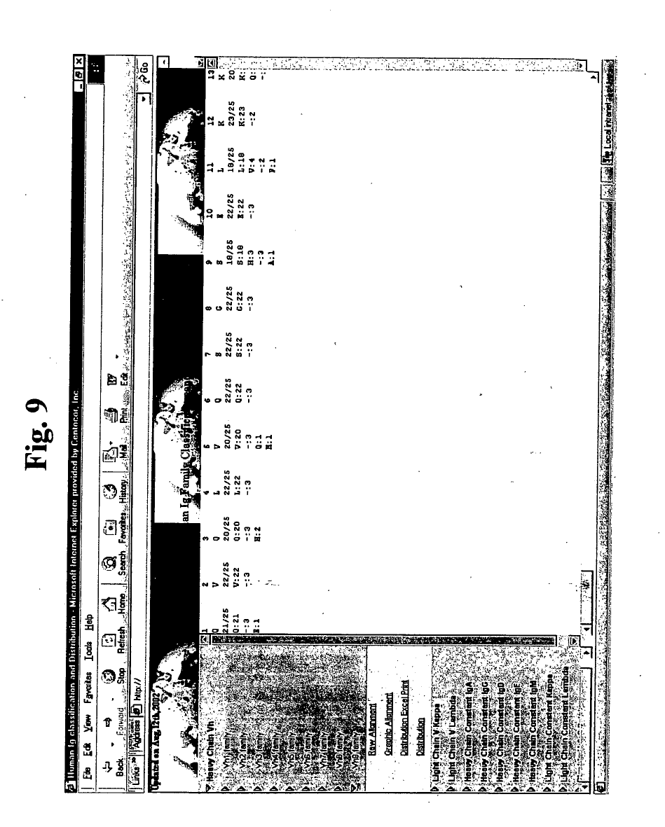

The third section displays calculated amino acid distribution statistics for each prototype positition identified by algorithm 3 in an alignment of subfamily sequences (FIG. 9). The first line indicates each numbered amino acid position of a prototype sequence. The second line shows the prototypical residue found in the numbered position. The third line indicates how many times the prototype amino acid occurs in a given position relative to the total number of sequences in the subfamily. The other lines display all other possible substitutions occurring at each prototype position and the number of times each substitution occurs in a given prototype position. These substitutions are sorted by their frequency of occurrence at each prototype position. The third section data display is also formatted for data import via a cut and paste function to programs such as Excel or Vector NTI. Data and formatting for the third section data display can be generated via algorithm 3.

Fourth Section Data Display

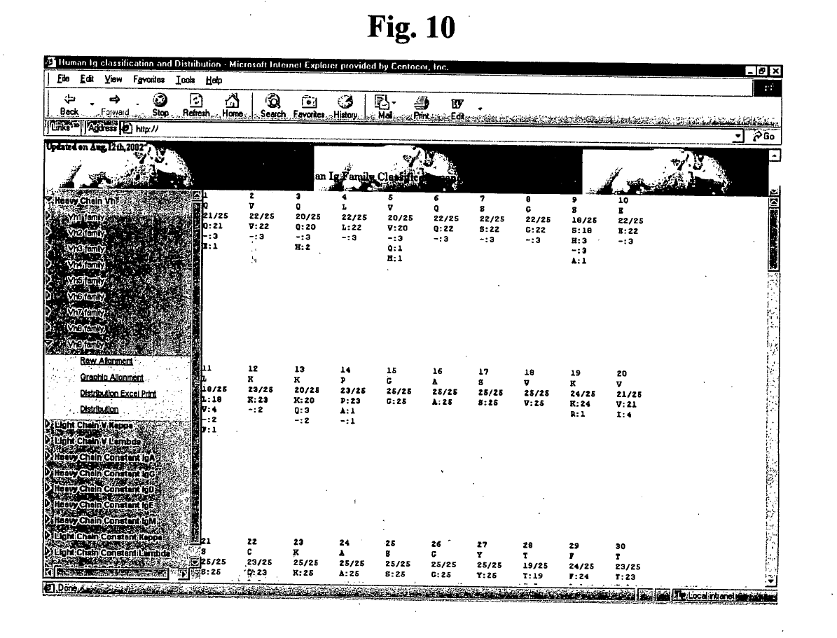

The fourth section data display is a distribution list for the Vh9 subfamily—which has the same contents as the section 3 display (FIG. 10). This section, however, is designed for easy web page display on a computer monitor and printing. To accomplish this, each line of the fourth section display shows substitution data for no more than 10 prototype amino acid positions. Annotations denoting the framework, CDR, CH1-4, and hinge regions may also added to the data displayed in this section or selected data from this section.

### EXAMPLE 1

### Use of the Database for Human Antibody Immunogenicity Prediction

The database of the invention can be used to predict whether a given antibody could be tolerated in humans without an adverse immune response. For example, a scientist wishing to determine if an antibody might generate an adverse response will obtain the sequences of the antibody's heavy and light chain variable and constant regions. The scientist will then use these heavy chain and light chain sequences to query the database through its BLAST searching feature. By reviewing the results of these BLAST searches the scientist can determine, for example, that the heavy and light chain variable regions of his antibody have a high level of similarity (e.g. >90% identical) to known human light and heavy chain variable region sequences. Most of the sequences differences occur in the CDRs; a minority occur in the frameworks. Similarly, the scientist can determine that the heavy and light chain constant regions are highly similar (e.g. >99% identical) to known human heavy and light chain constant region sequences. This information suggests to the scientist that the antibody is very human like and unlikely to generate an adverse immune response. This conclusion can be confirmed by performing a similar search in a database of mouse antibody sequences.

### EXAMPLE 2

### Use of the Database for Human Antibody Scaffold Alteration

In some instances it may be desirable to substitute a portion of one antibody with a portion of a second antibody. It may also be desirable to make amino acid substitutions. For example, a scientist may wish to replace a murine heavy chain variable region framework 1 with a human framework, or eliminate a post-translational modification site.

By using the BLAST feature of the invention a scientist could identify the human heavy chain variable region framework 1 region which is most similar to the murine framework 1 region to be substituted. The human framework 1 identified could then be substituted into the variable region of the antibody of interest.

Similarly, a scientist could eliminate a post-translational modification site in the constant region by using the BLAST feature of the database to determine which heavy chain constant region subfamily the antibody of interest belongs to. The scientist could then examine the Section 3 or 4 data displays for this subfamily and find the region corresponding to the post-translational modification site of interest. The scientist can then use the substitution frequency information for this region to select those substitutions which occur in the subfamily, but eliminate the post-translational modification site.

### REFERENCES

1. Johnson, G. and T. T. Wu. 2000. Kabat Database and its applications: future directions. Nucleic Acids Res. 29: 205-206.
2. Cook, G. P. and I. M. Tomlinson. 1995. The human immunoglobulin Vh repertoire. Immunology Today. 16: 237-242.
3. Knappik, A., Ge, L., Honegger, A., Pack, P., Fischer, M., Wellnhofer, G., Hoess, A., Wolle, J., Plückthun, A. and Virnekas, B. 2000. Fully synthetic human combinatorial antibody libraries (HuCAL) based on modular consensus frameworks and CDRs randomized with trinucleotides. J. Mol. Biol. 296: 57-86.
4. Kabat, E. A., Wu, T. T., Perry, H. M., Gottesman, K. S. and Foeller, C. 1991. Sequences of Proteins of Immunological Interest. 5^{th} edit, NIH publication no. 91-3242. US Department of Health and Human Services, Washington, D.C.
5. Altschul, Stephen F., Thomas L. Madden, Alejandro A. Schäffer, Jinghui Zhang, Zheng Zhang, Webb Miller, and David J. Lipman (1997), Gapped BLAST and PSI-BLAST: a new generation of protein database search programs. Nucleic Acids Res. 25:3389-3402.
6. Higgins D., Thompson J., Gibson T., Thompson J. D., Higgins D. G., and Gibson T. J. (1994), CLUSTAL W: improving the sensitivity of progressive multiple sequence alignment through sequence weighting, position-specific gap penalties and weight matrix choice. Nucleic Acids Res. 22:4673-4680.

What is claimed is:

1. A method for selecting, generating, comparing or analyzing human or human derived antibody or antibody fusion protein amino acid or nucleic acid sequences, comprising:
(a) accessing suitable antibody sequence databases and collecting constant, complimentarily determing regions (CDRs), and/or variable region sequences;
(b) subjecting the data collected in step (a) to Algorithm 1, wherein the sequences are classified into groups, superfamilies, and/or subfamilies;
(c) performing sequence alignment on all sequences assigned to a given subfamily in step (b);
(d) displaying subfamily multiple sequence alignment result of step (c);
(e) accessing antibody sequence databases and collecting variable region sequences;
(f) subjecting the data collected in step (e) to Algorithm 2, wherein the variable region sequences are classified into superfamilies and subfamilies;
(g) performing multiple sequence alignment on all sequences assigned to a given subfamily in step (f);
(h) displaying subfamily multiple sequence alignment results of step (g);
(i) subjecting the multiple sequence alignment data generated in step (c) or (g) to Algorithm 3, wherein each amino acid substitution is examined and the substitution's frequency of occurrence at a given position is calculated;
(j) determining the constant region subfamily prototype sequence and substitutions;
(k) displaying the the constant region subfamily prototype sequence and substitutions generated by step (j);
(l) determining the variable region subfamily prototype sequence and substitutions;
(m) displaying the variable region subfamily prototype sequence and substitutions generated by step (l);
(n) exporting the displayed results from step (d), (h), (k) or (m) to a web interface, wherein the displays can be viewed and BLAST searching can be performed.

2. Any invention described herein.
